(19) United States
(12) Patent Application Publication
(10) Pub. No.: US 2026/0106001 A1
(43) Pub. Date: Apr. 16, 2026

(54) MONITORING, MANAGEMENT, NOTIFICATIONS, AND PREDICTION PLATFORM FOR USER HEALTH STATUS

(71) Applicant: INTEGRAL DEVELOPMENT USA LLC, Miami, FL (US)

(72) Inventors: Jaime ARCE GARCÍA, Valledupar (CO); Hector ESTEBAN DÍAZ CABALLERO, Cartagena (CO)

(73) Assignee: INTEGRAL DEVELOPMENT USA LLC, Miami, FL (US)

(21) Appl. No.: 18/916,818

(22) Filed: Oct. 16, 2024

(51) Int. Cl. G16H 10/60; A61B 5/021; A61B 5/024; A61B 5/145; G08B 21/18; G16H 15/00; G16H 50/30

(52) U.S. Cl. CPC G16H 10/60; A61B 5/021; A61B 5/024; A61B 5/14542; G08B 21/182; G16H 15/00; G16H 50/30

(57) ABSTRACT

A platform for monitoring, management, notifications, and prediction of users'health status that obtains data from multiple devices of multiple variables in real-time. This platform integrates health data from multiple devices, including but not limited to portable devices and smart devices, medical devices, and clinical monitoring equipment, analyzes them in real-time using advanced algorithms, and provides automatic alerts to health professionals or end-users who wish to analyze the measured variables'information. The platform includes a company management module, a user management module, a roles and options configuration section, a user and/or patient control panel, a medical history or data management module, a reports and statistics module, an artificial intelligence and predictions module, and an emergency and external health services module, and security and privacy through multiple factors including a two-factor authentication and data encryption.

### FIELD OF THE DISCLOSURE

This invention falls within the technical field of platforms for monitoring, management, notifications, and prediction of user health status. Users can employ multiple high-precision devices to measure various vital signs or multiple variables in real-time. Some of these devices can be portable, such as smartwatches, exercise bands, rings, medical devices, or any peripheral device that can measure multiple variables in real-time associated with an individual. This information is collected and sent through a data transmission network that connects to an application, which allows the management of all devices simultaneously and uploads the information to the platform via an internet network connection. These data are subjected to a generative artificial intelligence, which allows predicting and notifying about the users'health status to health professionals and family members, activating immediate and emergency care protocols through geolocated health professional allies according to specialty.

### BACKGROUND OF THE INVENTION

The lack of monitoring, management, and prediction of people's health status through a platform that delivers notifications and allows tracking patients or users deteriorates the health status or even the quality of life of people because it does not allow analyzing the user's current state or the physical change resulting from the improvement of habits in physical conditions. Consequently, in many cases, it is not possible to diagnose adequately or in time the possible failure in the human body, nor to evidence the progress record according to the physical activity the user has had and generate early alerts for health professionals or notification protocols to family members so that they can deploy their actions.

Some solutions have been designed for monitoring users'health status, as evidenced in document US2017055851, which discloses a device containing a wireless sensor designed to monitor a patient's acceleration. The sensor includes multiple readings of physical variables such as temperature, ECG, an accelerometer, and a respiratory acoustic sensor, mounted on a substrate with openings to allow thermal and electrical communication. This device may also include a gyroscope and a wireless transceiver to transmit data such as temperature, ECG electrical signals, and movement. It is suitable for monitoring pressure ulcer and fall risks in patients. This device is limited because it only allows specific monitoring of diseases such as ulcers, thus being limited to users with this disease.

Similarly, document US2022238216 discloses a system that uses machine learning to optimize a device's operation based on the collection of biomarker data and feedback from a professional. The system adjusts its model to improve its performance, update its control, and provide risk assessments and notifications that reduce distractions while improving the model's quality. Additionally, it uses feedback to accelerate the learning process and adapt to changing situations for a better device outcome. This system is entirely dependent on a health professional to feed the artificial intelligence model, which limits the generation of real-time alerts as it depends on a human response and conditions the activity to a professional's criterion exclusively, who may not consider all the variables reported concerning the individual.

On the other hand, document US2016034663 discloses a method and a plurality of devices to assess an individual's health through data collection such as food intake, mood, vital signs, biological characteristics, genotype, lifestyle, and environment. These data are combined to generate a “health parameter” that reflects the subject's health status over a specific period. Additionally, it can detect health events, issue alerts, and compare the health of multiple subjects to prioritize medical attention. This health status assessment method does not allow interoperability between portable and medical devices, nor the storage of the user's medical history, nor does it implement an artificial intelligence model that supports the generation of personalized alerts that optimize the health professional's tasks through disease prediction. It also does not allow the integration of multiple devices into a single platform regardless of the devices'origin.

The proposed solutions in the state of the art do not address the need to develop platforms for monitoring, management, notifications, and prediction of users'health status that allow storing measurements from multiple devices of human body variables and that, in turn, serve to feed a generative artificial intelligence model so that the user can obtain data on their vital signs or information provided by multiple devices of multiple variables in real-time to perform an analysis with a plurality of specialists or end-users. The feeding of this information is done in a multi-device mode through the acquisition of multiple devices that the user can have in real-time, with information that is multi-variable, and with integration for data collection from all sources, regardless of the brand or type of information capture device, as long as the individual grants authorization as a user, which allows notifying about the users'health status to health professionals and family members, activating immediate and emergency care protocols through geolocated health professional allies according to specialty.

The object of the present invention is to provide a platform for monitoring, management, notifications, and prediction of users'health status where the information on vital signs or multiple variables of individuals is obtained by a plurality of multiple devices that perform real-time measurement. This information is sent through a data transmission network to an application that allows user management according to the number of devices required to link to monitor their health status. This application takes the data from multiple devices, which can be portable, smart, medical devices, installed in clinical settings, or of any nature, to be uploaded to the platform through a normalization method and, in turn, feed the artificial intelligence model and thus generate notifications about the user's health status through the platform for evaluation by a professional, specialist in the health area, or any end-user to allow an analysis of the information and have an efficient and immediate reaction in case of an emergency to be treated adequately and effectively without losing the patient's or user's health, physical condition, and life quality.

### SUMMARY

The present invention relates to a platform for monitoring, management, notifications, and prediction of users'health status that obtains data from multiple devices of multiple variables in real-time. This platform integrates health data from multiple devices, including but not limited to portable devices and smart devices, medical devices, and clinical monitoring equipment, analyzes them in real-time using advanced algorithms, and provides automatic alerts to health professionals or end-users who wish to analyze the measured variables'information. The platform includes a company management module, a user management module, a roles and options configuration section, a user and/or patient control panel, a medical history or data management module, a reports and statistics module, an artificial intelligence and predictions module, and an emergency and external health services module, and security and privacy through multiple factors such as two-factor authentication and data encryption.

The platform includes of several main components. First, data sampling and transmission are carried out through multiple portable devices, including but not limited to devices such as smartwatches, tracking rings, bracelets, and other wearables, smart devices, medical devices, and clinical monitoring equipment that capture required health data for real-time individual monitoring. Some of these are heart rate, blood oxygen levels, physical activity, and glucose, which can extend to all those variables that can be obtained through these devices, as long as they can transmit data directly through the Internet or indirectly via mobile phones or WiFi networks.

Secondly, data reception, standardization, and predictive analysis are carried out by a collector that receives and stores data in a RAW database. Subsequently, a standardization process organizes the data and stores them in a relational database. An artificial intelligence predictor analyzes the data in real-time, detects anomalous patterns, and generates predictive alerts.

The control panel for specialists is another component of the system. This user interface allows health professionals or end-users to review detailed data to analyze the human body's behavior, access users'medical history, and receive alerts according to their physical condition or health status. Additionally, a notifier sends automatic alerts via email, SMS, and any digital medium or instant messaging application.

The platform also includes functionalities for storing historical data, allowing the visualization of long-term health trends and the comparison of health metrics. Data security and privacy are ensured through data encryption both in transit and at rest, multi-factor authentication for platform access, access auditing to record who has accessed what data and when, and finally, interoperability and scalability integration to include functionalities for integration with other management systems of variables obtained through multiple devices of multiple variables in real-time.

The invention also incorporates an Allies App, which allows health service providers to receive notifications and apply for service provision according to geolocation and specialty. This application facilitates interaction with the system and improves the coordination of health services and emergency care with greater immediacy and traceability. On the other hand, it also allows the management of multiple devices, including but not limited to devices such as smartwatches, tracking rings, bracelets, and other wearables, smart devices, medical devices, and clinical monitoring equipment, which perform real-time measurement of multiple variables, as well as knowing the users'health status or making a preliminary diagnosis or physical condition according to what is obtained from the artificial intelligence model. This allies app also allows tracking the service provided by the professional managing the platform.

Key system functionalities include real-time monitoring, which allows capturing and analyzing health data in real-time and generating automatic alerts in case of detecting parameters outside normal ranges. The centralized control panel offers a detailed visualization of data and alerts in real-time, access to users'medical history, and generation of personalized reports. Predictive analysis uses artificial intelligence algorithms to detect anomalous patterns and foresee possible health complications. Data security and privacy are ensured through rigorous measures, and the system's interoperability and scalability are achieved through an open API for integration with other hospital management systems and medical devices, as well as the ability to handle a large volume of data and users simultaneously.

The benefits of this invention include proactive monitoring of multiple variables through multiple devices in real-time, not limited to those moments when the individual undergoes diagnostic tests in medical centers, which usually reflect only a fraction of the information. Therefore, the disclosed technology improves data quality and consequently increases the ability to prevent and respond to possible individual emergencies, directly promoting early interventions; optimizing medical work by centralizing and automating data collection and analysis, allowing monitoring of the user's physical progress who constantly exercises to improve their physical health, facilitating health professionals and users to analyze the behavior of measured variables and thus focus on not only critical cases but also continuous improvement and improving quality of life, providing continuous monitoring of vital signs and other health metrics.

In summary, this invention offers a comprehensive multi-platform solution for real-time health monitoring, combining data capture from multiple portable devices, including but not limited to devices such as smartwatches, tracking rings, bracelets, and other wearables, smart devices, medical devices, and clinical monitoring equipment, with a predictive analysis model and automatic alerts, all with a focus on data security and privacy.

### BRIEF DESCRIPTION OF THE DRAWINGS

FIG. 1: Shows the global architecture diagram of the platform.

FIG. 2: Shows the Information Flow Diagram where the user logs in and allows the upload of all information obtained from linked multiple devices and multiple variables.

FIG. 3: Shows the Specialist Control Panel Interface highlighting the following functionalities:

### Real-Time Monitoring

Visualization of critical health variables such as heart rate, blood oxygen levels, physical activity, and glucose, among others, according to the capture level of each device integrated into the platform.

### Patient List

A list of patients that can be filtered according to variables such as risk, diagnosis, and health status.

### Alarms and Alerts

Configurable alert system that notifies specialists when a health variable exceeds a critical threshold.

### Statistics and Analysis

Graphical visualization of monitored variables for each patient with trends over time and comparisons with historical data.

### Medical Interventions

Registration and recommendation of interventions based on alerts generated by the system.

FIG. 4: Shows the Allies App Interaction

### Notification Reception

Allies receive patient notifications according to their geolocation and specialty.

### Service Application

Allies can apply for health service provision.

### Real-Time Tracking

Allies can track the patient's evolution in real-time before arriving at the location.

### Geolocation

The Allies App provides the patient's geolocation to facilitate the route to the event location.

FIG. 5: Shows the management and operation diagram of the centralization of multiple devices and platform users.

These figures provide a detailed view of the components and functionalities of the platform for monitoring, management, notifications, and prediction of users'health status, highlighting its architecture, information flow, user interface, interaction with allies, security and privacy measures, and interoperability and scalability capabilities.

### DETAILED DESCRIPTION

The present invention relates to a platform for monitoring, management, notifications, and prediction of users'health status in real-time through multiple devices that measure multiple variables. This platform integrates health data from multiple devices, including but not limited to devices such as smartwatches, tracking rings, bracelets, and other wearables, smart devices, medical devices, and clinical monitoring equipment, analyzes them in real-time using advanced algorithms, and provides automatic alerts to health professionals. The platform includes a centralized control panel that allows the integration of professional health personnel, gyms, or any entity that requires knowing the health status of its users, secure storage of historical data, and a prediction module based on artificial intelligence.

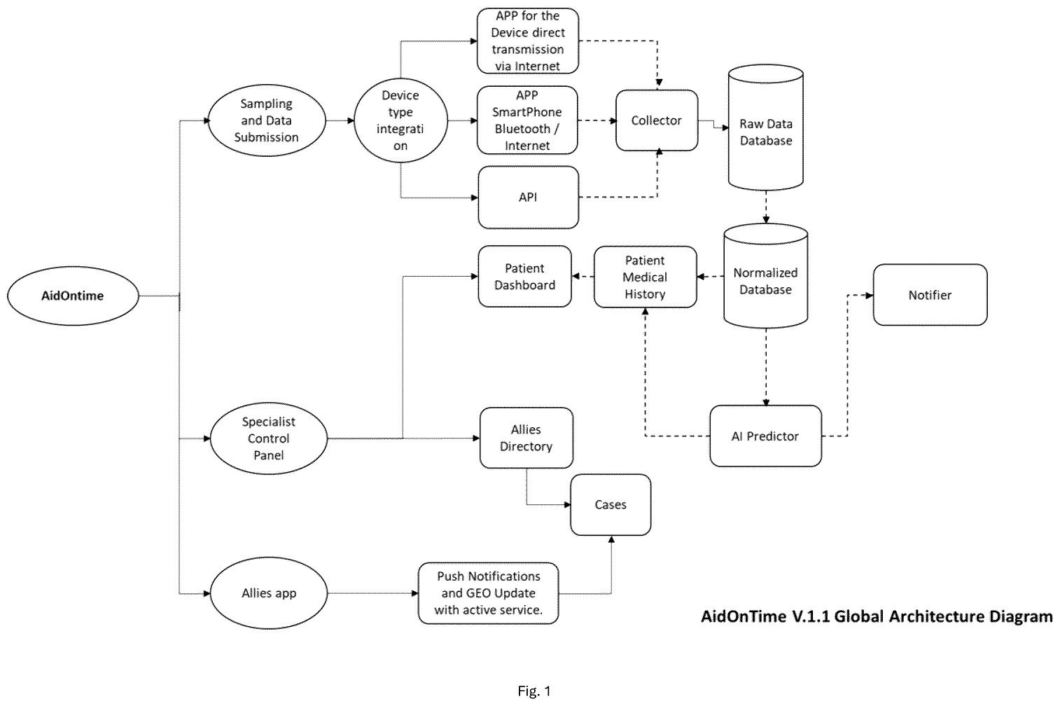

The platform for monitoring, management, notifications, and prediction of users'health status includes several main components. First, data sampling and transmission are carried out through multiple devices, including but not limited to devices such as smartwatches, tracking rings, bracelets, and other wearables, smart devices, medical devices, and clinical monitoring equipment that capture health data, including heart rate, blood oxygen levels, physical activity, and glucose, and any variable susceptible to measurement against the individual. These devices can transmit data directly through the Internet or indirectly via mobile phones or WiFi networks. FIG. 1 shows a system architecture diagram, divided into four main blocks: Data Sampling and Transmission, Data Reception, Standardization, and Predictive Analysis, Specialist Control Panel, and Allies App.

In the Data Sampling and Transmission block, portable devices continuously capture user health data. These devices may have different connectivity capabilities. Some devices allow direct data transmission through the Internet, either through an application installed on the device (Direct Connection) or through an API that transmits the data to a cloud platform (API Connection). Other devices do not have direct Internet connectivity and require an intermediary, such as a mobile phone, to transmit the data. These devices can connect to the mobile phone via Bluetooth and use an application to temporarily store the data and then transmit it to a centralized server when an Internet connection is available (Indirect Transmission).

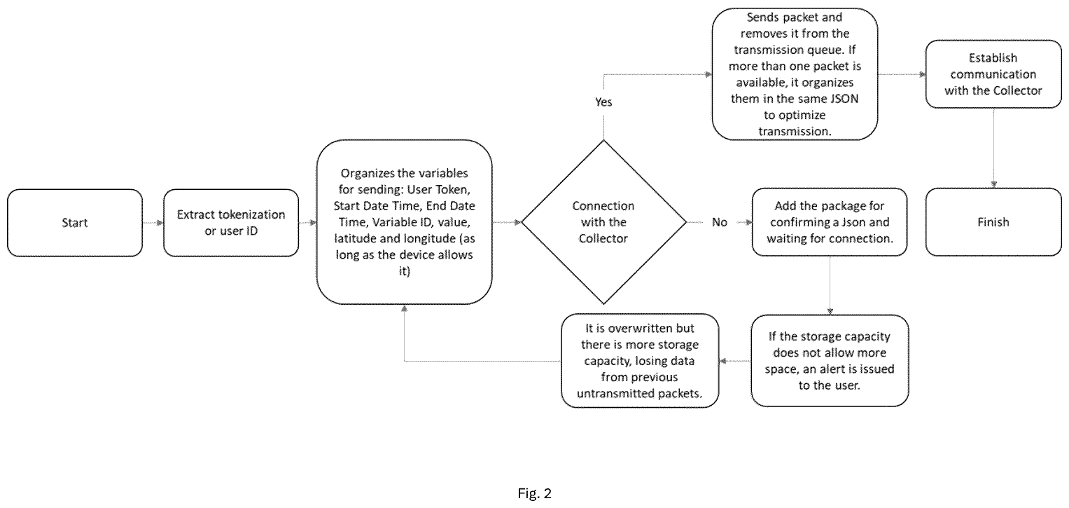

Secondly, data reception, standardization, and predictive analysis are carried out by a collector that receives and stores data in a RAW database. The collector activates when it receives data from portable devices and quickly stores them in a non-relational database to avoid any processing delay. Subsequently, a standardization process organizes the data and stores them in a relational database. This process includes indexing the data by user, date, and time, and other relevant indexes to facilitate data query and analysis. An artificial intelligence predictor analyzes the data in real-time, detects anomalous patterns, and generates predictive alerts. FIG. 2 illustrates the information flow from data capture by portable devices to alert generation and visualization in the control panel. This flow includes data reception by the collector, standardization and storage in a relational database, and predictive analysis using artificial intelligence algorithms.

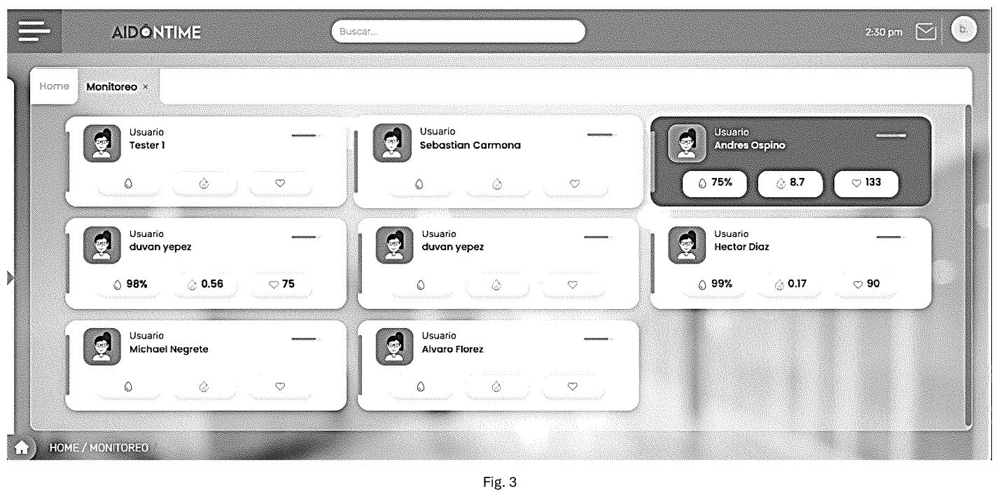

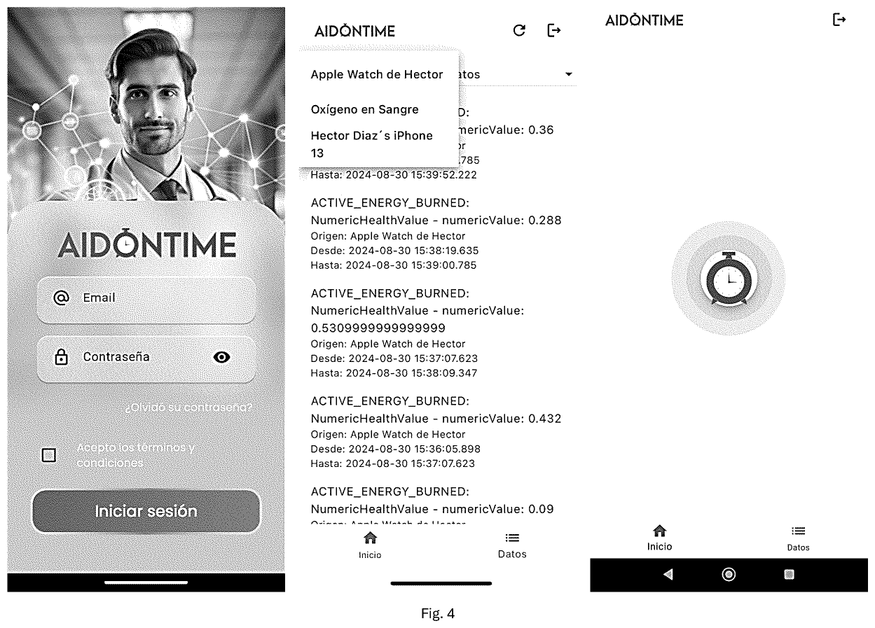

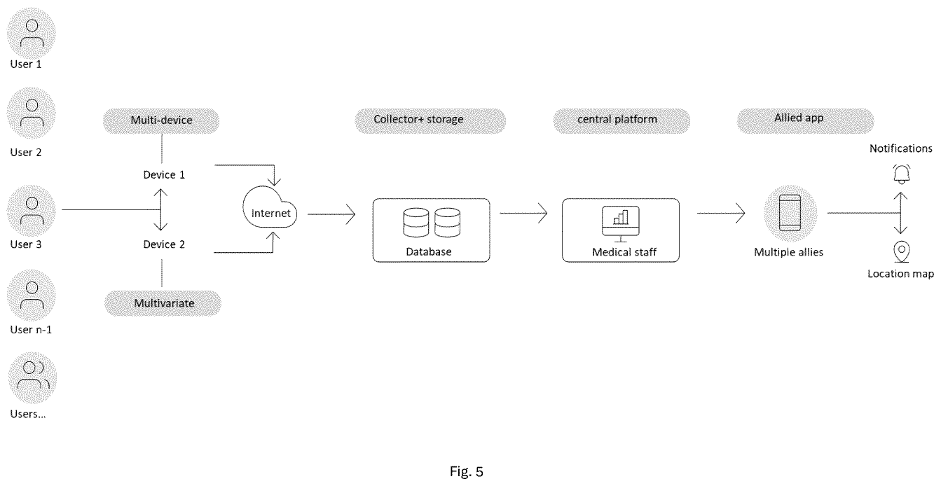

The control panel for specialists is another crucial component of the system. This user interface allows health professionals to review detailed data, access users'medical history, and receive alerts according to their specialty and geolocation. The control panel is designed to be intuitive and customizable, allowing specialists to configure the metrics and variables they wish to monitor prominently. Additionally, a notifier sends automatic alerts via email, SMS, and Instant Messaging Systems. FIG. 3 shows the specialist control panel interface, highlighting functionalities such as real-time monitoring of critical health variables, a filterable patient list according to variables such as risk and diagnosis, a configurable alert system, graphical visualization of health trends, and medical intervention registration.

The system also includes functionalities for storing historical data, allowing the visualization of long-term health trends and the comparison of health metrics. Historical data is stored in a normalized relational database, allowing efficient indexing and querying of data. Health professionals can use these functionalities to compare a patient's current health status with their history and with data from other patients with similar characteristics. Data security and privacy are ensured through data encryption both in transit and at rest, multi-factor authentication for platform access, and access auditing to record who has accessed what data and when.

The invention also incorporates an Allies App, which allows health service providers to receive notifications and apply for service provision according to geolocation and specialty. This application facilitates interaction with the system and improves the coordination of health services. Allies can receive notifications of patients requiring attention and apply to provide services based on their specialty and geographic location. The Allies App also allows health service providers to track the patient's evolution in real-time before arriving at the event location, using geolocation to facilitate the route to the event location. FIG. 4 illustrates the interaction of the Allies App with the platform for monitoring, management, notifications, and prediction of users'health status, showing how allies receive notifications, apply for services, track the patient's evolution in real-time, and use geolocation to facilitate the route to the event location.

Key system functionalities include real-time monitoring, which allows capturing and analyzing health data in real-time and generating automatic alerts in case of detecting parameters outside normal ranges. The centralized control panel offers a detailed visualization of data and alerts in real-time, access to users'medical history, and generation of personalized reports. Predictive analysis uses artificial intelligence algorithms to detect anomalous patterns and foresee possible health complications. These algorithms are trained using historical data and continuously improved through machine learning, optimizing recommendations and alerts for each patient.

Data security and privacy are ensured through rigorous measures, and the system's interoperability and scalability are achieved through an open API for integration with other hospital management systems and medical devices, as well as the ability to handle a large volume of data and users simultaneously.

The benefits of this invention include proactive monitoring, which improves the ability to respond to possible emergencies and promotes early interventions; optimizing medical work by centralizing and automating data collection and analysis, allowing health professionals to focus on critical cases; and improving quality of life by providing continuous monitoring of vital signs and other health metrics, thus improving users'quality of life.

FIG. 5 shows the user and device management flow diagram, where information from multiple devices is collected and sent to be stored and to feed the artificial intelligence prediction model to generate the necessary notifications for user care. These functions allow scalability and administration from any ally or entity that requires knowing the health status of its users.

In summary, this invention offers a comprehensive solution for real-time health monitoring, combining data capture from multiple portable devices that perform real-time measurement of multiple variables, predictive analysis, and automatic alerts, all with a focus on data security and privacy.

What is claimed is:

1. A platform for monitoring, management, notifications, and prediction of a users'health status comprising:
multiple portable devices for capturing multiple variables in real-time, including portable devices, smart devices, medical devices, and clinical monitoring equipment, which send information to an allies'application;
a data reception and standardization module that organizes and stores a data in at least two relational, non-relational raw, and normalized databases to be displayed on a specialists'panel;
an artificial intelligence predictor for data analysis and real-time alert generation fed by the normalized database;
a notifier at an output of the artificial intelligence predictor;
a centralized control panel for health professionals, allowing detailed data review, access to users'medical history, and generation of personalized reports.

2. The platform for monitoring, management, notifications, and prediction of users'health status according to claim 1, wherein the multiple devices capture real-time health data of the individual, including but not limited to:
heart rate;
blood oxygen levels;
physical activity;
glucose levels; and
blood pressure.

3. The platform for monitoring, management, notifications, and prediction of users'health status according to claim 1, where the data reception and standardization module comprise:
a collector that receives data from portable devices and initially stores them in a RAW database;
a standardization process that organizes the data and stores them in a relational database;
an AI predictor that analyzes the data and generates predictions based on the user's history and detected patterns.

4. The platform for monitoring, management, notifications, and prediction of users'health status according to claim 1, where the artificial intelligence predictor uses supervised and unsupervised learning algorithms to:
detect anomalous patterns in health data;
generate automatic alerts in case of detecting parameters outside normal ranges; provide predictions about possible health complications.

5. The platform for monitoring, management, notifications, and prediction of users'health status according to claim 1, where the centralized control panel allows users managing the information to:
review detailed user data in real-time;
access users'medical history;
generate and visualize personalized reports; and
receive critical alert notifications through various channels such as any instant messaging application, email, or SMS.

6. A method for real-time health monitoring integrated into the platform for monitoring, management, notifications, and prediction of users'health status comprising:
capturing health data through portable devices, smart devices, medical devices, and clinical monitoring equipment;
transmitting the data to a centralized server;
analyzing the data in real-time through an artificial intelligence predictor;
generating automatic alerts and notifying users managing the information of individuals with multiple devices.

7. The method according to claim 6, where the captured health data include, but are not limited to:
heart rate;
blood oxygen levels;
physical activity;
glucose levels; and
blood pressure.

8. The method according to claim 6, where data transmission is carried out through:
direct Internet connection from portable devices;
indirect connection through mobile phones or WiFi networks.

9. The method according to claim 6, where real-time data analysis comprises:
data reception by a collector;
standardization and storage of data in a relational database;
predictive analysis using artificial intelligence algorithms.

10. The method according to claim 6, where automatic alerts are sent through any instant messaging application or email.

11. The method according to claim 6, where historical data are stored and organized to allow:
visualization of long-term health trends;
comparison of health metrics between different users and time periods.

12. The method according to claim 6, where security and privacy measures are implemented comprising:
data encryption both in transit and at rest;
multi-factor authentication for platform access;
access auditing to record who has accessed what data and when.

13. The platform for monitoring, management, notifications, and prediction of users'health status according to claim 1, where the centralized control panel includes functionalities for:
management of types of companies that may be involved, such as hospitals, clinics, health centers, gyms, or any entity that wishes to know the health status of its users;
user management with defined profiles, roles, and permissions;
configuration of services offered by each registered company;
real-time monitoring of critical health variables of patients;
generation of detailed medical reports on patients'health status.

14. The platform for monitoring, management, notifications, and prediction of users'health status according to claim 1, where the artificial intelligence-based prediction module comprises:
real-time anomaly detection;
predictions based on medical history and predictive models;
machine learning to optimize recommendations and alerts.

15. The platform for monitoring, management, notifications, and prediction of users'health status according to claim 1, where the emergency and external health services module comprises:
integration with emergency services to automatically contact the nearest health services to the patient via GPS;
notification to authorized family members in critical situations;
real-time tracking of the patient's evolution before the arrival of emergency services.

16. The platform for monitoring, management, notifications, and prediction of users'health status according to claim 1, where data security and privacy are guaranteed through:
data encryption using advanced protocols;
multi-factor authentication for platform access;
access auditing to record and monitor data access.

17. The platform for monitoring, management, notifications, and prediction of users'health status according to claim 1, where interoperability and scalability are achieved through:
multiple user devices;
internet connection;
passage through the collector and storage in the database;
the central platform and the application and integration of multiple allies.

18. The platform for monitoring, management, notifications, and prediction of users'health status according to claim 1, where the platform includes an Allies App that allows integration and is not limited to the following functions:
management of multiple devices that perform real-time measurement of multiple variables;
receiving user status notifications through their geolocation and thus prioritizing the nearest possible specialist or entity;
anticipating disease diagnosis or physical condition through the artificial intelligence predictor;
tracking the service provided by the professional managing the platform.
